(19) United States
(12) Patent Application Publication
(10) Pub. No.: US 2009/0270736 A1
(43) Pub. Date: Oct. 29, 2009

(54) CAPSULE-TYPE MEDICAL APPARATUS

(75) Inventors: Shinichi MIYAMOTO, Tokyo (JP); Yoshiyuki OKUNO, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/496,843

(22) Filed: Jul. 2, 2009

Related U.S. Application Data
(63) Continuation of application No. PCT/JP2007/050044, filed on Jan. 5, 2007.

(51) Int. Cl. A61B 8/14

(52) U.S. Cl. 600/462

(57) ABSTRACT

A capsule-type medical apparatus includes a capsule to be introduced into a tubular body cavity, and a string member that extends from one end of the capsule and is able to be pulled when the capsule is pulled. The capsule is provided with a shunting section for shunting blocking substances that impede the movement of the capsule when the string member is pulled to pull the capsule

This application is a continuation application of PCT/JP2007/050044 filed on Jan. 5, 2007, the entire contents of which are incorporate herein by this reference.

### BACKGROUND OF INVENTION

1. Field of the Invention

The present invention relates to a capsule-type medical apparatus having a capsule to be introduced into a tubular cavity such as a body cavity from which a string member is extended.

2. Description of the Related Art

Conventionally, a capsule-type medical apparatus for introducing a capsule configured for a medical treatment into a body cavity to collect information of a lesion and/or administration of medical fluid have been known. Also, in recent years, a capsule endoscope is being implemented as a capsule-type medical apparatus for introducing a capsule into a body cavity to acquire images in the body cavity. A medical capsule for ultrasound diagnosis disclosed in Japanese Patent Application Laid-Open Publication No. 2-224650 is an example of a capsule-type medical apparatus in the field of ultrasound observation apparatuses in which ultrasound signals for observation are sent to vital tissues and echo signals reflected from the vital tissues are received to acquire ultrasound tomographic images for diagnosis.

The medical capsule for ultrasound diagnosis includes transmission means for converting the echo signals from a subject that are received by an ultrasound transducer provided in the capsule into electric signals and delivering the electric signals to an external apparatus outside of the body. The transmission means has a wireless or wired configuration.

The wired capsule-type medical apparatus does not need to have a wireless communications device or battery incorporated in a capsule thereof, which facilitates the downsizing of the capsule as compared to a wireless capsule-type medical apparatus. In addition, the wired capsule-type medical apparatus provides advantages that the positions of the capsule introduced in a body cavity can be changed by pulling a signal cable for example, or the body cavity can be observed while the signal cable is pulled to draw back the capsule, the signal cable being a string member that also functions as a pull member extended from the capsule.

### SUMMARY OF THE INVENTION

A capsule-type medical apparatus of the present invention includes a capsule to be introduced into a tubular body cavity, and a string member that extends from one end of the capsule and is able to be pulled when the capsule is pulled. The capsule is provided with a shunting section for shunting blocking substances. When the string member is pulled to pull the capsule, blocking substances that impede the movement of the capsule is shunted by the shunting section.

### BRIEF DESCRIPTION OF THE DRAWINGS

FIG. 1 is a view illustrating a configuration of a medical capsule for an ultrasound diagnosis apparatus and the main parts of an ultrasound capsule;

FIG. 2 is a view illustrating a configuration of an outer surface of the ultrasound capsule;

FIG. 3 is a cross sectional view taken along the III-III line of FIG. 2 and illustrating a cross sectional shape of a groove;

FIG. 4 is a view illustrating an operation of grooves provided in the outer surface of the ultrasound capsule;

FIG. 5 is a view illustrating a state where a part of blocking substances piled on the operator's side of the ultrasound capsule is shunted through the grooves to the proximal end side;

FIG. 6 is a view illustrating another configuration of the grooves provided in the outer surface of the capsule;

FIG. 7 is a view illustrating another configuration of the grooves provided in the outer surface of the capsule;

FIG. 8 is a view illustrating further another configuration of the grooves provided in the outer surface of the capsule;

FIG. 9 is a view illustrating a configuration of an ultrasound capsule provided with a spiral cover;

FIG. 10 is a view illustrating an operation of a spiral cover provided to the ultrasound capsule;

FIG. 11 is a view illustrating a configuration of an ultrasound capsule having two drive motors;

FIG. 12 is a view showing a state where blocking substances are piled on the operator's side of the ultrasound capsule;

FIG. 13 is a view illustrating the operation of an ultrasound capsule;

FIG. 14 is a view illustrating a cross sectional configuration of a balloon;

FIG. 15 is a view illustrating the ultrasound capsule having the balloon;

FIG. 16 is a view illustrating an operation of the balloon, and a cross sectional view taken along the XVI-XVI line of FIG. 15;

FIG. 17 is a cross sectional view illustrating a configuration of a signal cable;

FIG. 18 is a view illustrating another configuration of a multi-lumen tube;

FIG. 19 is a view illustrating a configuration of an ultrasound capsule having a multi-lumen that includes two fluid ducts as a signal cable;

FIG. 20 is a view illustrating a signal cable having an observation apparatus connector;

FIG. 21 is a view illustrating a connection between the signal cable and the ultrasound observation apparatus; and

FIG. 22 is a view illustrating another configuration of an ultrasound capsule apparatus.

### DETAILED DESCRIPTION OF THE PREFERRED EMBODIMENT(S)

Now, with reference to the drawings, several embodiments of the present invention will be described in detail below.

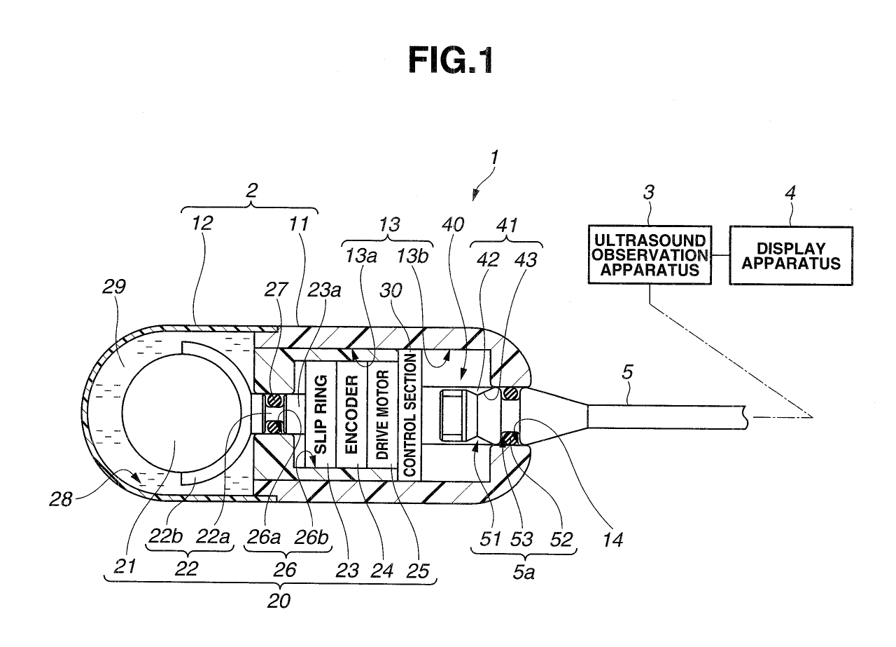

With reference to FIG. 1 to FIG. 8, a first embodiment of the present invention will be described below.

As shown in FIG. 1, a medical capsule for ultrasound diagnosis apparatus of the present embodiment (hereinafter, simply referred to as an ultrasound capsule apparatus) 1 includes an ultrasound capsule 2 for example, as a capsule-type medical apparatus. The ultrasound capsule apparatus 1 is configured with the ultrasound capsule 2, an ultrasound observation apparatus 3 that is an external apparatus, and a display apparatus 4. The ultrasound capsule 2 is a capsule to be introduced into a body cavity, and has a proximal end portion from which a signal cable 5 is extended, the signal cable 5 being a string member that also functions as a pull member. The signal cable 5 electrically connects the ultrasound capsule 2 to the ultrasound observation apparatus 3. That is, in the ultrasound capsule apparatus 1 of the present embodiment, the signal transmission between the ultrasound observation apparatus 3 and the ultrasound capsule 2 is performed by wired transmission means. The ultrasound observation apparatus 3 is connected to the display apparatus 4 by a video cable.

The ultrasound capsule 2 is configured with a capsule body 11 and a transducer cover 12. The transducer cover 12 is generally cylindrical, and has end portions that are configured to have curved surfaces, for example generally semi-spherical surfaces. The transducer cover 12 is secured to the distal end portion of the capsule body 11 in a liquid-tight manner.

The capsule body 11 is provided with a space 13 for a unit mounting hole 13a and a connector chamber 13b, and a cable hole 14 communicating between the space 13 and the outside. Into the cable hole 14, a capsule connector 5a provided to the signal cable 5 is removably installed. That is, the signal cable 5 is removable to the ultrasound capsule 2. The capsule body 11 also has end portions that are configured to have curved surfaces.

The ultrasound capsule 2 has an ultrasound unit 20, a control section 30, and a cable removal mechanism section 40 arranged therein.

The ultrasound unit 20 is configured with an ultrasound transducer 21 that is of a mechanical scan type for example, a transducer securing member 22 having a transducer shaft 22a, a slip ring 23 that is rotary signal transfer means, an encoder 24, a drive motor 25, and a unit body 26. The unit body 26 is provided with a hole 26a and an axial bore 26b communicating between the hole 26a and the outside. The unit body 26 has an outer peripheral surface that is integrally secured at a predetermined position in the space 13 by adhesive for example, in a watertight manner.

The hole 26a of the unit body 26 is a unit mounting hole. In the hole 26a, the slip ring 23, the encoder 24, and the drive motor 25 are received. The drive motor 25 has a motor shaft (not shown) extended therefrom. The motor shaft has a distal end portion to which the transducer shaft 22a is axially supported at the proximal end of the transducer shaft 22a via a ball bearing 23a that is fixedly attached to the slip ring 23.

The transducer shaft 22a has a distal end portion to which a securing portion 22b is provided. The securing portion 22b is arranged on the outside of the unit body 26. The securing portion 22b is integrated with the ultrasound transducer 21. The transducer shaft 22a has an O-ring 27 arranged on the middle thereof. The O-ring 27 is closely held against the inner peripheral surface of the axial bore 26b by a predetermined pressure. This allows the ultrasound transducer 21 to be arranged in a space defined in a liquid tight manner by the inner peripheral surface of the transducer cover 12, the distal end surface of the capsule body 11, the distal end surface of the unit body 26, and the O-ring 27. The space 28 is filled with an ultrasound transfer medium 29 such as water.

The ultrasound transducer 21 has an input/output signal cable (not shown) extended therefrom. The ultrasound transducer 21 and the control section 30 are electrically connected to each other via the input/output signal cable and the like.

The control section 30 is configured with a substrate for example, and is disposed on the proximal end side with respect to the ultrasound unit 20 in the unit mounting hole 13a. In other words, the control section 30 is disposed on the cable removal mechanism section 40 side. The substrate is provided with a rotation detection circuit, an ultrasound sending/receiving circuit, a signal process circuit, an output circuit and the like which are not shown. The rotation detection circuit is electrically connected to the encoder 24 to detect a rotation state of the ultrasound transducer 21. The ultrasound sending/receiving circuit sends/receives ultrasound signals to/from the ultrasound transducer 21 via the slip ring 23. The signal process circuit processes echo signals received at the ultrasound sending/receiving circuit, for example. The output circuit outputs ultrasound data of ultrasound observation images that are processed and generated by the signal process circuit to the ultrasound observation apparatus 3.

The space on the proximal end side in the unit mounting hole 13a provides the connector chamber 13b. The connector chamber 13b is provided with a cable removal mechanism section 40. The cable removal mechanism section 40 has a quick disconnect 41 to which the capsule connector 5a is removably connected, the capsule connector 5a being provided at the distal end portion of the signal cable 5 extended from the ultrasound observation apparatus 3. The quick disconnect 41 is electrically connected and integrated to the substrate that constitutes the control section 30. The quick disconnect 41 is provided with a pair of engaging pawls 42 that face to each other and constitute a connection. The engaging pawls 42 protrude on the proximal end side of the connector chamber 13b. The engaging pawls 42 are provided with a projection 43.

Into the quick disconnect 41, the capsule connector 5a is removably installed via the cable hole 14. The capsule connector 5a is provided with engaging recesses 51 corresponding to the engaging pawls 42 as a coupling. With the capsule connector 5a being connected to the quick disconnect 41, the engaging pawls 42 are engaged with engaging recesses 51 at projections 43 of the engaging pawls 42. The confirmation of the engagement is afforded by a click feeling in the installation. The engaged installation produces a predetermined pressure which makes the O-ring 52 closely contact with the inner peripheral surface of the cable hole 14. The O-ring 52 is disposed in a circumferential groove 53 around the cylindrical portion of the capsule connector 5a.

The quick disconnect 41 is provided with a plurality of pins (not shown) for example. Correspondingly, the capsule connector 5a is provided with pin holes in which the pins of the quick disconnect 41 are individually engaged. The pressure welding between the pins of the quick disconnect 41 and the pin holes in the capsule connector 5a causes the signal cable 5 to be connected to the ultrasound capsule 2.

The coupling strength between the ultrasound capsule 2 and the signal cable 5 is set based on the connection strength between the quick disconnect 41 and the capsule connector 5a, the coupling strength at the coupling, and the slide resistance of the O-ring 52. That is, in the present embodiment, the coupling strength is set to be a desired value based on the relationships between the outer diameter of each pin and each pin hole, the length for pressure welding, or the relationship between the projection 43 and the engaging recesses 51 obtained through experiments and simulations. The specific coupling strength is set to be smaller than the tensile strength that causes damage to the signal cable 5 when pulled.

Meanwhile, the ultrasound observation apparatus 3 is provided with an image processor for ultrasound observation, an amplifier circuit, a transmission circuit and the like which are not shown. The transmission circuit is connected with a signal line (not shown) that is inserted through the signal cable 5. The transmission circuit processes ultrasound data inputted via the signal line, or drive signals outputted via the signal line, for example.

Specifically, the drive signals from the transmission circuit of the ultrasound observation apparatus 3 go through the signal line in the signal cable 5, the capsule connector 5a, the cable removal mechanism section 40, the control section 30, a second input signal cable (not shown), a metal brush (not shown), the ring portion (not shown) of the slip ring 23 the metal brush contacts, and a first input signal cable (not shown), to the ultrasound transducer 21. To the contrary, the echo signals from the ultrasound transducer 21 are transferred through the ultrasound transducer 21, a first output signal cable (not shown), the ring portion (not shown) of the slip ring 23, the metal brush (not shown) that electrically contacts the ring portion, a second output signal cable, the cable removal mechanism section 40, the capsule connector 5a, and the signal line in the signal cable 5, to the transmission circuit of the ultrasound observation apparatus 3.

The amplifier circuit at least amplifies the ultrasound data. The image processor for ultrasound observation generates video signals of a B mode image, a Doppler image, an image by harmonic imaging and the like based on the ultrasound data, and outputs the signals to the display apparatus 4. This allows the display apparatus 4 to display various ultrasound tomographic images on a screen.

The ultrasound observation apparatus 3 monitors every signal transmitted through the transmission circuit, and the monitored result is used to determine the attachment/detachment of the signal cable 5 to the ultrasound capsule 2. When determining that the capsule connector 5a is detached from the ultrasound capsule 2, the ultrasound observation apparatus 3 turns off the outputting of all of the signals to the ultrasound capsule 2. In the turning off, the ultrasound observation apparatus 3 outputs a notice signal of the OFF state to the display apparatus 4, so that a letter(s) or an image notifying that the detachment of the signal cable from the ultrasound capsule is displayed on the screen of the display apparatus 4, for example.

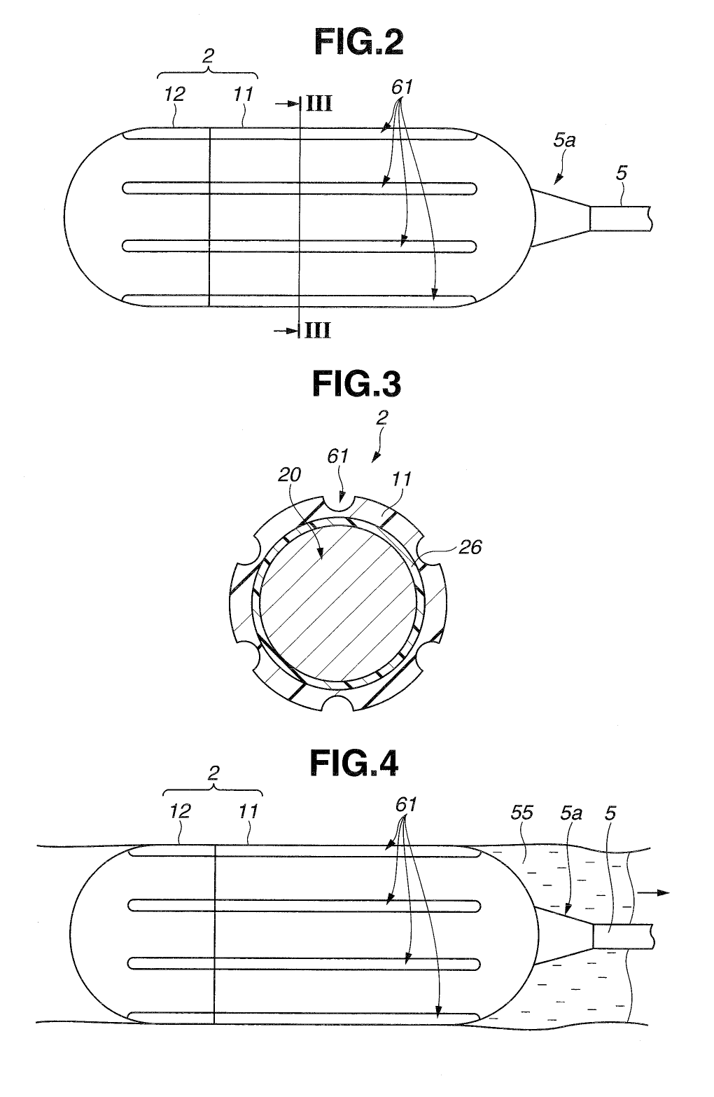

As shown in FIG. 2, the outer surface of the capsule body 11 and the transducer cover 12 both constituting the ultrasound capsule 2 are provided with a plurality of grooves 61 generally parallel to the longitudinal direction thereof at uniform intervals in the circumferential direction. Each of the grooves 61 is a shunting section that allows the blocking substances to shift from one side to the other side thereof, the substances being piled mucus, digestive fluid, water, residue and the like and providing resistance against movement. Each of the grooves 61 has ends that terminate at a curved surface of the capsule body 11 and a curved surface of the transducer cover 12. As shown in FIG. 3, the grooves 61 have a semi-circular cross section, for example.

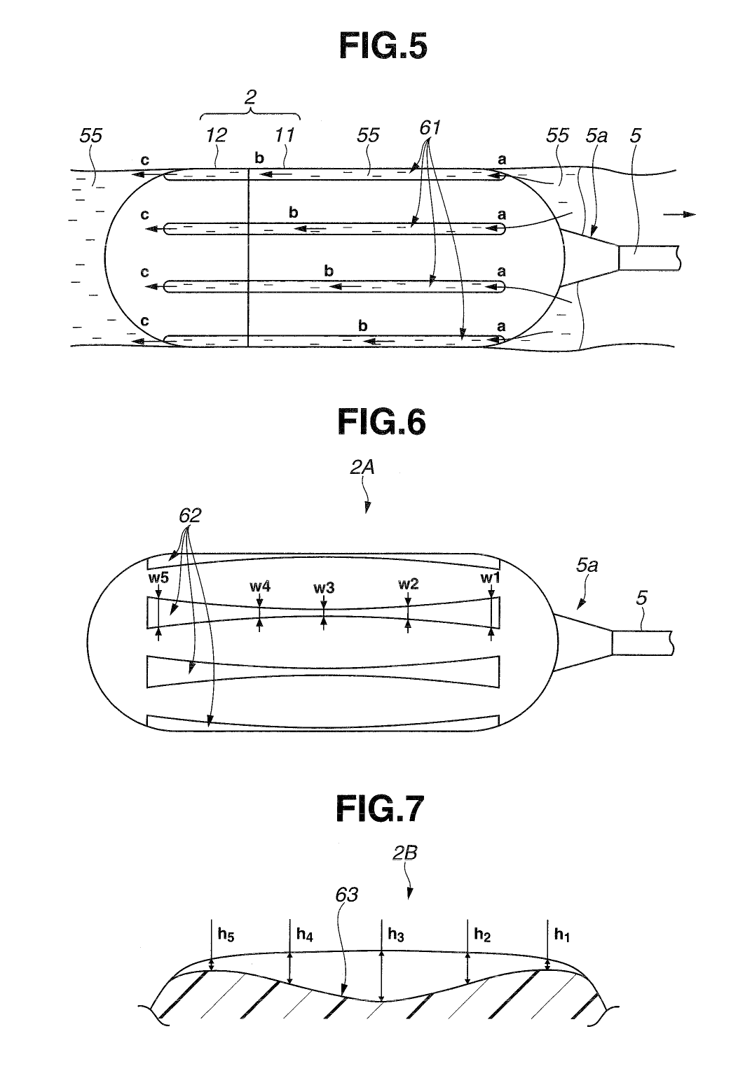

With reference to FIG. 1, FIG. 4, and FIG. 5, the operations of the ultrasound capsule apparatus 1 having the ultrasound capsule 2 that is configured as described above will be described below.

An operator prepares the ultrasound capsule 2 for an ultrasound observation of the part around esophagus or small intestine of a subject for example. Then the operator couples the capsule connector 5a of the signal cable 5 extended from the ultrasound observation apparatus 3 to the cable removal mechanism section 40 of the ultrasound capsule 2. In the coupling, the engaging pawls 42 and the engaging recesses 51 are engaged with each other giving a click feeling to the operator.

The subject for the test swallows the ultrasound capsule 2 prepared by the operator. The swallowed ultrasound capsule 2 passes through the esophagus and stomach due to the peristaltic motion, and reaches the small intestine for example. In the movement, the operator uses the ultrasound observation apparatus 3 that is an external apparatus to output drive signals to the ultrasound capsule 2. Then the ultrasound transducer 21 emits ultrasound, and the ultrasound transducer 21 receives ultrasound echoes. The ultrasound echoes received by the ultrasound transducer 21 are transmitted as ultrasound data from the ultrasound transducer 21 to the ultrasound observation apparatus 3 via the control section 30 and the signal cable 5 as described above.

The ultrasound observation apparatus 3 generates video signals from the transmitted ultrasound data for an ultrasound diagnosis, and outputs the video signals to the display apparatus 4. This allows the operator to observe the ultrasound tomographic image displayed on the screen of the display apparatus 4 for an ultrasound diagnosis.

The operator pulls the signal cable 5 to draw the ultrasound capsule 2 to the operator's side when another ultrasound diagnosis is necessary for a site the ultrasound capsule 2 has already passed, when an observation is made while the ultrasound Capsule 2 is being drawn back, or when the ultrasound capsule 2 is taken out through the oral cavity after the ultrasound diagnosis is completed.

As shown in FIG. 4, as the ultrasound capsule 2 is drawn back, the mucus, digestive fluid, residue, water and the like attached to the wall of the small intestine for example are gradually piled up on the proximal end side of the ultrasound capsule 2, which forms the blocking substances 55 that impedes the movement of the ultrasound capsule 2.

Subsequently, the operator further pulls the signal cable 5 to draw the ultrasound capsule 2 to the operator's side. Then as shown in FIG. 5, a part of the blocking substances 55 piled on the proximal end side of the ultrasound capsule 2 enters the grooves 61 along the curved surface as shown by the arrows a as the ultrasound capsule 2 moves. A further drawing of the ultrasound capsule 2 to the operator's side causes parts of the blocking substances 55 to continuously enter the grooves 61. This makes the blocking substances 55 that have already entered the grooves 61 urged to pass through the grooves 61 to the distal end sides thereof as shown by the arrows b. After that, the blocking substances 55 passing through the grooves 61 are shunted to distal end side of the ultrasound capsule 2 as shown by the arrows c.

This allows the ultrasound capsule 2 to be smoothly drawn to the operator's side without being impeded by the blocking substances 55 piled on the proximal end side of the ultrasound capsule 2.

In this way, in the ultrasound capsule apparatus for wired signal transmission by connecting the ultrasound observation apparatus to an ultrasound capsule by a signal cable, the ultrasound capsule has an outer surface provided with grooves that are parallel to each other in the longitudinal direction thereof. The configuration allows the piled mucus, digestive fluid, residue, water and the like on the proximal end side of the ultrasound capsule to be shunted on the distal end side of the ultrasound capsule through the grooves as the ultrasound capsule is drawn to the operator's side when the signal cable is pulled to draw the ultrasound capsule to the operator's side. Therefore, in the ultrasound capsule apparatus of the present embodiment, in the pull of the signal cable to retract the ultrasound capsule, any impediment to the movement of the ultrasound capsule by blocking substances can be prevented.

As a result, in the ultrasound capsule apparatus of the present, the movement of the ultrasound capsule in the direction opposite to the direction in accordance with peristaltic motion is facilitated when the signal cable is pulled. This enables a smooth recheck of a site the ultrasound capsule has already passed, a smooth observation while the ultrasound capsule is being drawn back, a smooth collection of the ultrasound capsule through oral cavity, and the like.

The mucus, digestive fluid, residue, water and the like piled on the operator's side of the ultrasound capsule are shunted on the distal end side as the ultrasound capsule is moved to the operator's side, which prevents an excess tension applied to the signal cable due to the blocking substances.

When the signal cable 5 is pulled to move the ultrasound capsule 2 in the direction opposite to the direction in accordance with peristaltic motion, the blocking substances 55 piled on the proximal end side of the ultrasound capsule 2 may not be shunted to the distal end side through the grooves 61. In the case, the blocking substances 55 impede the movement of the ultrasound capsule 2 to the operator's side. This causes a tension equal to or more than the predetermined coupling strength to be applied to the coupling between the capsule connector 5a and the cable removal mechanism section 40. Then the capsule connector 5a is detached from the quick disconnect 41. That is, the signal cable 5 is detached from the ultrasound capsule 2.

This makes the signal cable 5 rapidly collected through the mouth of the patient without cutting various signal lines through the signal cable 5. While, the ultrasound capsule 2 detached from the signal cable 5 moves in accordance with peristaltic motion, and is naturally discharged through the anus. That is, in pulling the signal cable 5 to retract the ultrasound capsule 2, when the blocking substances 55 impedes the movement of the ultrasound capsule 2, any pain to the patient and any cutting of signal lines through the signal cable 5 can be prevented.

In addition, the described above pins on the quick disconnect 41 side and the pin holes on the capsule connector 5a side realize the configuration in which no pin contacts a tubular body cavity when the signal cable 5 is detached from the ultrasound capsule 2 in a body cavity. If the pins have round tips, the quick disconnect 41 may be provided with pin holes and the pins may be arranged on the capsule connector 5a.

Furthermore, in the above described embodiment, the grooves 61 in the ultrasound capsule 2 individually have a semicircular cross section. And the grooves 61 are arranged generally parallel to each other in the longitudinal direction thereof in the outer surface of the capsule. The configuration of the grooves as shunting sections in the outer surface of the ultrasound capsule 2, however, is not limited to this. That is, the ultrasound capsule may be configured with the grooves shown in FIG. 6 to FIG. 8.

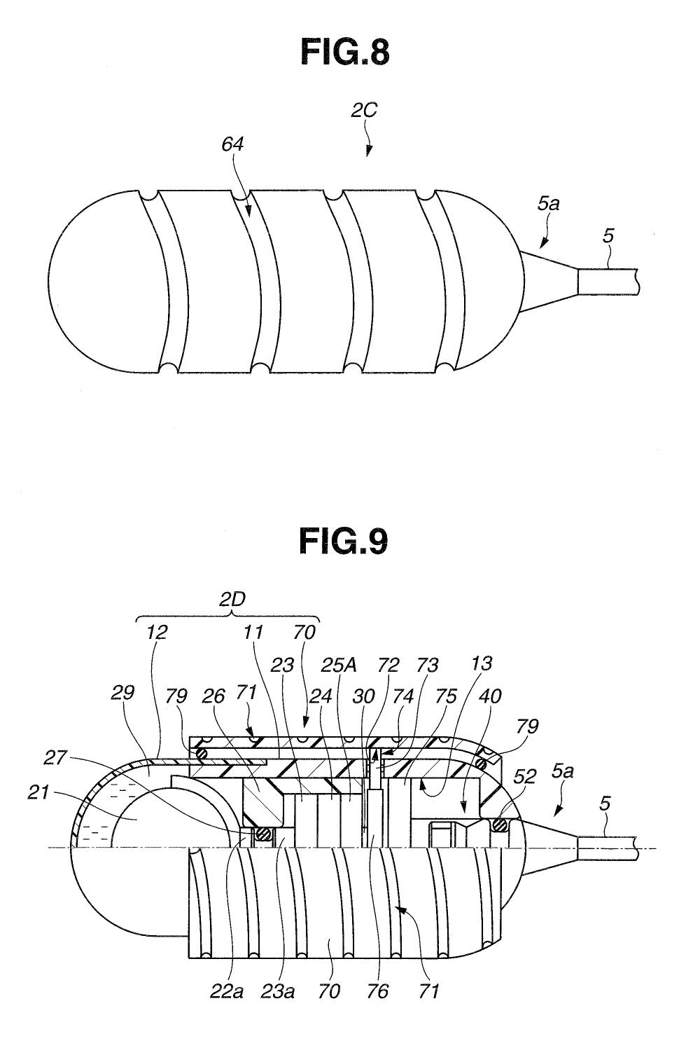

An ultrasound capsule 2A shown in FIG. 6 is provided with grooves 62 as shunting sections that have continuously changing width dimensions in the outer surface of the capsule. An ultrasound capsule 2B shown in FIG. 7 is provided with grooves 63 as shunting sections that have continuously changing depth dimensions in the outer surface of the capsule. An ultrasound capsule 2C shown in FIG. 8 is provided with spiral grooves 64 as shunting sections in the outer surface of the capsule.

Each of the grooves 62 of the ultrasound capsule 2A shown in FIG. 6 is set to have larger groove widths w1 and w5 on the ends thereof and a smaller groove width w3 at the center thereof, for example. This allows a part of the blocking substances piled on the proximal end side of the ultrasound capsule 2A to smoothly enter the grooves 62 having the larger groove width w1. The entered blocking substances are gradually piled in the grooves 62 on the groove width w3 side through the grooves 62 having the groove width w2. As the piled amount is increased, the piled blocking substances in the grooves 62 around the part having the groove width w3 are urged out of the grooves 62 having the groove width w3 to move to the distal end side through the grooves 62 having the groove widths w4 and w5.

Each of the grooves 63 of the ultrasound capsule 2B shown in FIG. 7 is set to have smaller grove depths h1 and h5 on the ends thereof and a larger groove depth w3 at the center thereof, for example. This allows the blocking substances that entered the grooves 63 having the grove depth h1 to be gradually piled on the grove depths h3, h4, and h5 side through the grooves 63 having the grove depth h2. As the piled amount is increased in the grooves 63, the blocking substances are urged out of the grooves 63 having the grove depth h5 to the distal end side.

The grooves 62 and 63 of the above described embodiment have continuously changed groove width dimensions or groove depth dimensions, but may have the dimensions that at least partially change in steps. Alternatively, the ultrasound capsule may be configured with the grooves provided in the capsule surface to have groove width dimensions and groove depth dimensions that can be conveniently changed.

Each of the spiral grooves 64 of FIG. 8 has a spiral angle or a spiral shape that can be conveniently changed so that the blocking substances piled on the proximal end side can be smoothly shunted on the distal end side, and the mucus, digestive fluid, residue and the like attached to walls can be removed. This causes the blocking substances to move to the distal end side through the spiral grooves 64. The grooves 64 may also have the groove width dimensions or groove depth dimensions that can be changed as described above.

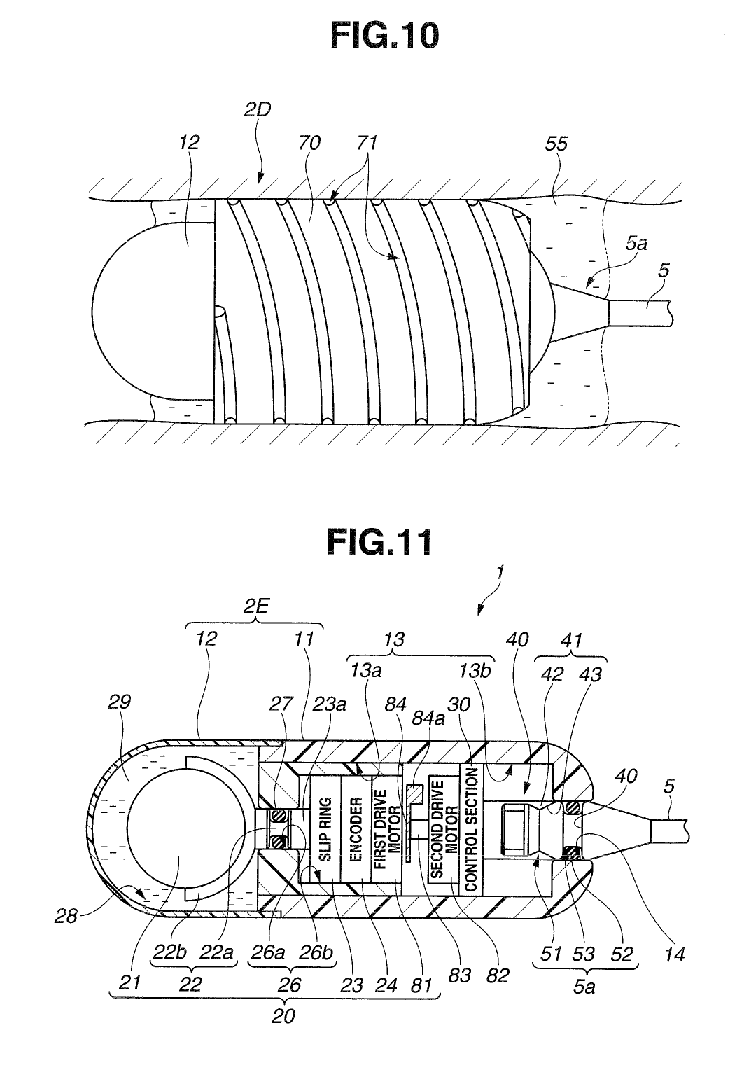

With reference to FIG. 9 and FIG. 10, a second embodiment of the present invention will be described below.

As shown in FIG. 9, an ultrasound capsule 2D of the present embodiment includes a spiral cover 70 as a shunting section on the outside of a capsule that is configured with a capsule body 11 and a transducer cover 12. The spiral cover 70 is rotatably arranged to the capsule via O-rings 79. The spiral cover 70 has a generally tubular shape, and is provided with a spiral groove 71 that has a semi-circular cross section in the outer surface of the spiral cover 70. The spiral cover 70 is provided with a tooth portion 72 around the entire circumference thereof at a predetermined position on the inner peripheral surface. The tooth portion 72 is configured to mesh with a first gear 73 provided on the capsule body 11. In the present embodiment, four first gears 73 are circumferentially provided at uniform intervals.

In the present embodiment, the ultrasound unit 20 is provided with a drive motor 25A, instead of the drive motor 25. The drive motor 25A has a motor shaft extended on the distal end side thereof (not shown), and a motor shaft 75 extended on the proximal end side thereof. The motor shaft on the distal end side has a distal end portion to which the transducer shaft 22a is axially supported at the proximal end thereof via a ball bearing 23a that is fixedly attached to the slip ring 23, as in the case of the above embodiment. The motor shaft 75 on the proximal end side axially supports a second gear 76 that meshes with the first gear 73.

In the above configuration, a driving of the drive motor 25A causes the motor shaft on the distal end side and the motor shaft 75 on the proximal end side to rotate. The rotation of the motor shaft on the distal end side causes the ultrasound transducer 21 to rotate. And the rotation of the motor shaft 75 causes the second gear 76 to rotate. Because the second gear 76 meshes with the first gears 73, the rotation of the second gear 76 results in the rotation of the first gear 73. Because the first gears 73 meshes with the tooth portion 72, the spiral cover 70 rotates in the direction opposite to the rotation of the motor shaft 75.

The first gear 73 is arranged in a notch hole portion 74 that communicates between the space 13 and the outside.

The O-rings 79 are disposed in circumferential grooves (not shown) that are provided in the circumferences of the capsule body 11 and the transducer cover 12 and have a predetermined cross sectional shape, and in circumferential grooves (not shown) that are provided in the inner peripheral surface of the spiral cover 70 and have a predetermined cross sectional shape. This seals the inner surface of the spiral cover 70 and the outer surface of the capsule in a water-tight manner when the spiral cover 70 is arranged to the outer surface of the capsule via the O-rings 79. Therefore, any entrance of water to the swallowed ultrasound capsule 2D or body fluid of a body cavity into the space with the hole 74 can be prevented.

Other configurations are similar to those of the first embodiment, and the same members are designated with the same reference numerals, which will not be described below.

The operations of the ultrasound capsule 2D configured as described above will be described below.

An operator prepares the ultrasound capsule 2D for an ultrasound observation of the part around esophagus or small intestine of a subject for example. Then the operator couples the capsule connector 5a of the signal cable 5 extended from the ultrasound observation apparatus 3 to the cable removal mechanism section 40 of the ultrasound capsule 2D.

A subject for the test swallows the ultrasound capsule 2D prepared by the operator. The swallowed ultrasound capsule 2 passes through the esophagus and stomach due to the peristaltic motion, and reaches the small intestine for example. In the movement, the operator uses the ultrasound observation apparatus 3 that is an external apparatus to output a drive signal to the ultrasound capsule 2. Then the drive motor 25A is driven and the ultrasound transducer 21 starts to rotate in a predetermined direction to emit ultrasound. The spiral cover 70 rotates in the direction opposite to the direction in which the ultrasound transducer 21 rotates. In the rotation, the close contact between the spiral groove 71 and an intestinal wall or the like produces an impelling force. That is, the ultrasound capsule 2D is advanced to a deeper part with the help of the impelling force.

The ultrasound echoes received by the ultrasound transducer 21 are transmitted as ultrasound data from the ultrasound transducer 21 to the ultrasound observation apparatus 3 via the control section 30 and the signal cable 5 as described above. The ultrasound observation apparatus 3 generates video signals from the transmitted ultrasound echoes for an ultrasound diagnosis, and outputs the video signals to the display apparatus 4. This allows the operator to observe the ultrasound tomographic image displayed on the screen of the display apparatus 4 for an ultrasound diagnosis.

The operator pulls the signal cable 5 to draw the ultrasound capsule 2D to the operator's side when another ultrasound diagnosis is necessary for a passed site or when the ultrasound capsule 2 is taken out through the oral cavity after the ultrasound diagnosis is completed. As shown in FIG. 10, as the ultrasound capsule 2 is drawn back, the mucus, digestive fluid, residue and the like are gradually piled up on the proximal end side of the ultrasound capsule 2D, which forms the blocking substances 55 that may impede the movement of the capsule.

Thus, the operator uses the observation apparatus 3 to output a drive signal to the ultrasound capsule 2D while pulling the signal cable 5 to draw the ultrasound capsule 2D to the operator's side. Then the drive motor 25A is driven and the ultrasound transducer 21 starts to rotate, and also the spiral cover 70 starts to rotate. Because the direction in which the spiral cover 70 rotates is opposite to the direction for an ultrasound observation, in addition to the pulling force by the signal cable 5, an impelling force of the spiral cover 70 causes the ultrasound capsule 2D to move toward the operator's side. In the movement, the mucus, digestive fluid, residue, water and the like attached to the wall of a small intestine for example are forcefully shunted to the distal end side of the ultrasound capsule 2D through the spiral groove 71 provided in the rotating spiral cover 70. Therefore, as shown in FIG. 10, the blocking substances 55 is not piled on the proximal end side of the ultrasound capsule 2D, but shunted on the distal end side of the ultrasound capsule 2D through the spiral groove 71. This allows the ultrasound capsule 2D to be smoothly drawn to the operator's side without being impeded by the blocking substances 55 when the signal cable is pulled.

In this way, in the ultrasound capsule apparatus for wired signal transmission by connecting the ultrasound observation apparatus to an ultrasound capsule by a signal cable, the ultrasound capsule has an outer surface provided with a rotatable spiral cover. The spiral cover is configured to rotate when the signal cable is pulled to draw the ultrasound capsule to the operator's side. The configuration allows the substances such as mucus, digestive fluid, residue and the like attached to a wall of small intestine to be forcefully shunted to the distal end side through the groove provided in the rotating spiral cover while the ultrasound capsule is drawn to the operator's side. Therefore, in the ultrasound capsule apparatus of the present embodiment, in the pull of the signal cable to retract the ultrasound capsule, the piling of the mucus, digestive fluid, residue, water and the like on the operator's side of the ultrasound capsule can be prevented. As a result, the operation to retract the ultrasound capsule can be smoothly performed.

In the above described embodiment, one drive motor 25A is provided to drive both of the ultrasound transducer 21 and the spiral cover 70. Two drive motors in the capsule, however, may be provided to drive the ultrasound transducer 21 and the spiral cover 70 individually. The latter configuration allows only the ultrasound transducer 21 to rotate during an ultrasound observation.

Also, when the ultrasound capsule is configured with two drive motors provided thereto, one of the drive motors is for the ultrasound transducer and the other drive motor is for the spiral cover. The other drive motor, however, is not limited to be for the spiral cover, and the second drive motor 82 may be for an oscillation generating apparatus that constitutes a shunting section like an ultrasound capsule 2E shown in FIG. 11, for example.

With reference to FIGS. 11 to 13, a third embodiment of the present invention will be described below.

The ultrasound capsule 2E of the present embodiment is, as shown in FIG. 11, provided with a first drive motor 81 and a second drive motor 82. The first drive motor 81 operates similarly to the above described drive motor 25, and causes the ultrasound transducer 21 to rotate.

To the contrary, the second drive motor 82 has a motor shaft 83 to which a weight 84 is integrally provided, the weight 84 having a generally L-shape and including an eccentric portion 84a. A driving of the second drive motor 82 causes the motor shaft 83 to rotate, which also causes the weight 84 to rotate. The rotation of the weight 84 having the eccentric portion 84a changes the position of the center of gravity of the ultrasound capsule 2E, resulting in a predetermined oscillation of the ultrasound capsule 2E. That is, the second drive motor 82 and the weight 84 provided to the motor shaft 83 of the drive motor 82 constitute an oscillation generating apparatus. The other configurations are similar to those of the first embodiment, and the same members are designated with the same reference numerals, which will not be described below.

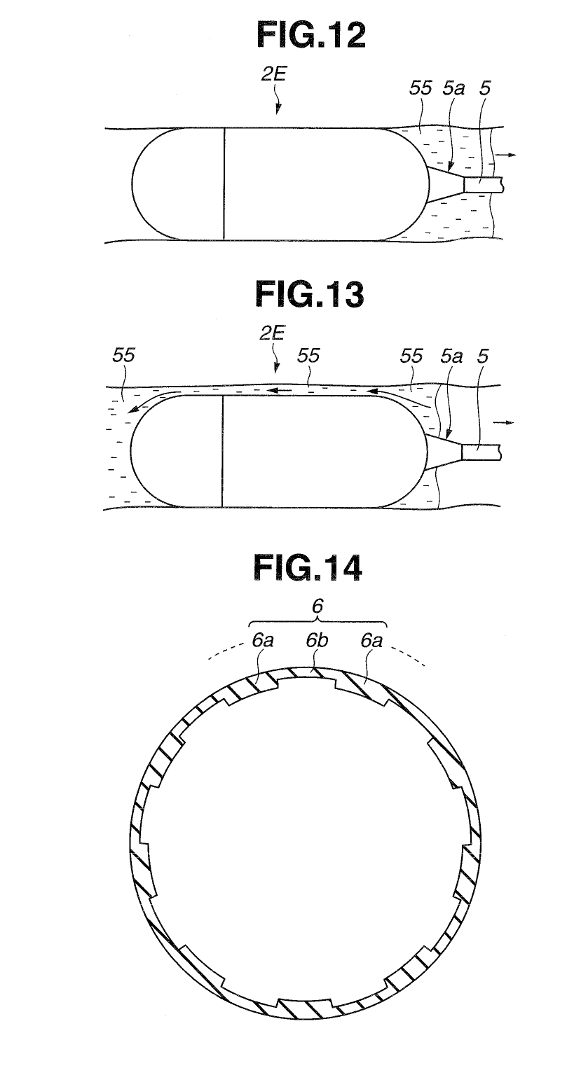

As for the ultrasound capsule 2E of the present embodiment, the operator pulls the signal cable 5 when another ultrasound diagnosis is necessary for a passed site or when the ultrasound capsule 2E is taken out through the oral cavity after an ultrasound diagnosis is completed, for example. As shown in FIG. 12, as the ultrasound capsule 2E is drawn back, the mucus, digestive fluid, residue and the like attached to a wall of small intestine for example are gradually piled up on the proximal end side of the ultrasound capsule 2E, which forms the blocking substances 55. Thus the movement of the ultrasound capsule 2E is impeded even when the signal cable 5 is pulled.

Then the operator causes the second drive motor 82 to be driven via the ultrasound observation apparatus 3, and pulls the signal cable 5 again. This causes the motor shaft 83 of the second drive motor 82 to rotate, and the weight 84 to rotate, resulting in a predetermined oscillation of the ultrasound capsule 2E. The oscillation produces a gap between the outer surface of the ultrasound capsule 2E and the wall.

When the ultrasound capsule 2E is drawn to the operator's side while the ultrasound capsule 2E is oscillating, as shown in FIG. 13, a part of the blocking substances 55 piled on the proximal end side of the ultrasound capsule 2E is shunted to the distal end side of the ultrasound capsule 2E through the gap produced between the ultrasound capsule 2E and the wall due to the oscillation. This allows the ultrasound capsule 2E to be smoothly drawn to the operator's side without being impeded by the blocking substances 55 piled on the proximal end side of the ultrasound capsule 2E.

In the present embodiment, as shown in FIG. 12, while the blocking substances 55 is piled on the proximal end side of the ultrasound capsule 2E, the second drive motor 82 is driven to cause the ultrasound capsule 2E to oscillate. An operator, however, may cause the second drive motor 82 to be driven to oscillate the ultrasound capsule 2E when pulling the signal cable 5 to draw the ultrasound capsule 2E to the operator's side. This allows the substances such as mucus, digestive fluid, and residue attached to a wall of small intestine to be forcefully shunted through the gap to the distal end side as the oscillating ultrasound capsule 2E moves to the operator's side.

In this way, in the ultrasound capsule apparatus for wired signal transmission by connecting the ultrasound observation apparatus to an ultrasound capsule by a signal cable, two drive motors are provided in the ultrasound capsule. One of the drive motors is for an ultrasound transducer, and the other drive motor is for an oscillation generating apparatus. When a signal cable is pulled to draw the ultrasound capsule to the operator's side or when the blocking substances on the proximal end side of the ultrasound capsule impedes the movement, the motor for an oscillation generating apparatus is driven to cause the ultrasound capsule to oscillate. Then the oscillation of the ultrasound capsule produces a gap between the outer surface of the ultrasound capsule and a wall, so that the mucus, digestive fluid, residue, water and the like are shunted to the distal end side through the gap as the ultrasound capsule is moved to the operator's side.

Therefore, in the ultrasound capsule apparatus of the present embodiment also, in the pull of the signal cable to retract the ultrasound capsule, any impediment to the movement of the ultrasound capsule by the mucus, digestive fluid, residue, water and the like can be prevented.

With reference to FIG. 14 to FIG. 16, a fourth embodiment of the present invention will be described below.

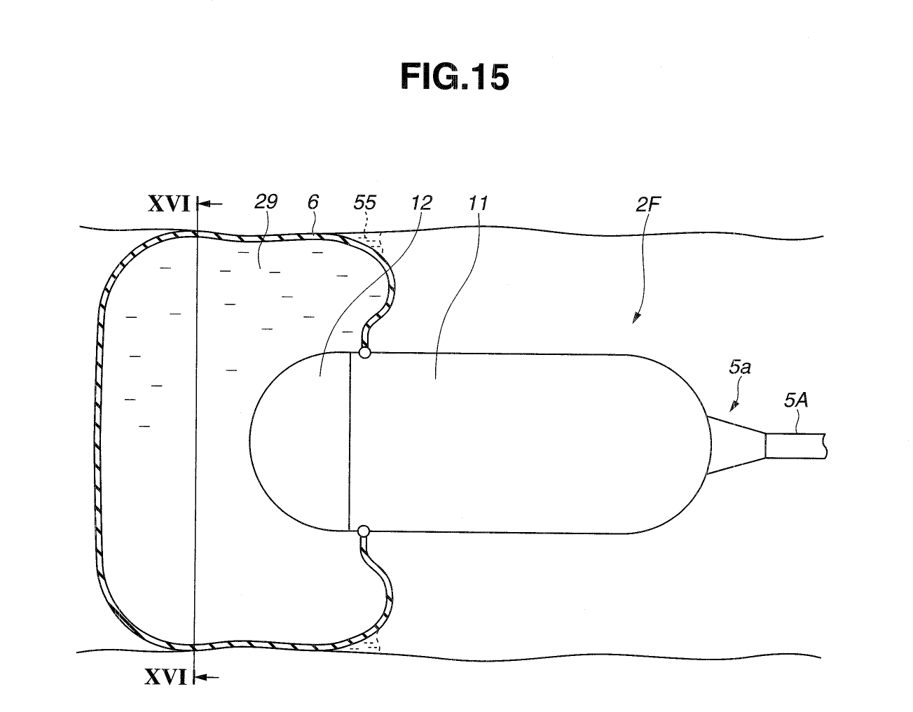

An ultrasound capsule 2F of the present embodiment is provided with a balloon 6 at a predetermined position, as shown in FIG. 15. The balloon 6 is filled with an ultrasound transfer medium 29. The ultrasound capsule 2F is provided with a duct for ultrasound transfer medium (not shown) for supplying the ultrasound transfer medium 29 to the balloon 6. The duct for ultrasound transfer medium is configured to have one end in communication with the balloon 6, and the other end in communication with a fluid duct (not shown) that is provided in a signal cable 5A.

The ultrasound observation apparatus 3 of the present embodiment is separately or integrally provided with a pump for supplying the ultrasound transfer medium 29 and a tank for storing the ultrasound transfer medium 29. The balloon 6 is configured to inflate or deflate under the control of a control section (not shown) in the ultrasound observation apparatus 3.

As shown in FIG. 14, the balloon 6 is made of an inflatable/deflatable elastic member having ultrasound transparency. The balloon 6 is provided with a plurality of thicker portions 6a and thinner portions 6b that constitute shunting sections. The thicker portions 6a are formed to have a predetermined thickness dimension, and the thinner portions 6b are formed to have a predetermined thickness dimension smaller than the thickness dimension of the thicker portions 6a by a predetermined amount. The thicker portions 6a and the thinner portions 6b are elongated in the longitudinal axial direction, and are alternately arranged in the circumferential direction. In the balloon 6 having the above configuration, the thinner portions 6b have an elasticity smaller than the elasticity of the thicker portions 6a. Therefore, the thinner portions 6b are readily deformed by a less power as compared to the thicker portions 6a.

The other configurations are similar to those of the first embodiment, and the same members are designated with the same reference numerals, which will not be described below.

An operator prepares the ultrasound capsule 2F for an ultrasound observation of the part around esophagus or small intestine of a subject for the test for example. Then the operator couples the capsule connector 5a of the signal cable 5A extended from the ultrasound observation apparatus 3 to the cable removal mechanism section 40. This makes the fluid duct and the duct for ultrasound transfer medium being in communication with each other. The operator attaches the balloon 6 at a predetermined position on the ultrasound capsule 2F. Then the operator checks the presence/absence of bubbles via the ultrasound observation apparatus 3 after the ultrasound transfer medium 29 is supplied to the balloon 6. When bubbles are present in the balloon 6, the bubbles are removed from the balloon 6 while the ultrasound transfer medium 29 is being supplied. When the removal of the bubbles is completed, the balloon 6 is deflated.

A subject for the test swallows the ultrasound capsule 2F that is prepared by the operator and has the deflated balloon 6. The swallowed ultrasound capsule 2F passes through the esophagus and stomach due to the peristaltic motion, and reaches the small intestine for example. After determining that the ultrasound capsule 2F has reached a target site in the small intestine, the operator uses the ultrasound observation apparatus 3 that is an external apparatus to output a drive signal to the ultrasound capsule 2F so that a predetermined amount of the ultrasound transfer medium 29 is supplied to the balloon 6.

Then, as shown in FIG. 15, the balloon 6 closely contacts a wall of the small intestine, and also the drive motor 25 is driven to cause the ultrasound transducer 21 to emit ultrasound. Then as described above, the ultrasound echoes received by the ultrasound transducer 21 are transmitted as ultrasound data from the ultrasound transducer 21 to the ultrasound observation apparatus 3 via the control section 30 and the signal cable 5.

The ultrasound observation apparatus 3 generates video signals from the transmitted ultrasound data for an ultrasound diagnosis, and outputs the video signals to the display apparatus 4. This allows the operator to observe the ultrasound tomographic image displayed on the screen of the display apparatus 4 for an ultrasound diagnosis.

The operator pulls the signal cable 5 to draw the ultrasound capsule 2F to the operator's side when another ultrasound diagnosis is necessary for a site the ultrasound capsule 2F has already passed. As shown in FIG. 15, because the balloon 6 of the ultrasound capsule 2F is in close contact with a wall, as the ultrasound capsule 2F is drawn back, the mucus, digestive fluid, residue, water and the like attached to the wall are gradually piled up on the operator's side of the balloon 6, which forms the blocking substances 55 that may impede the movement of the balloon 6.

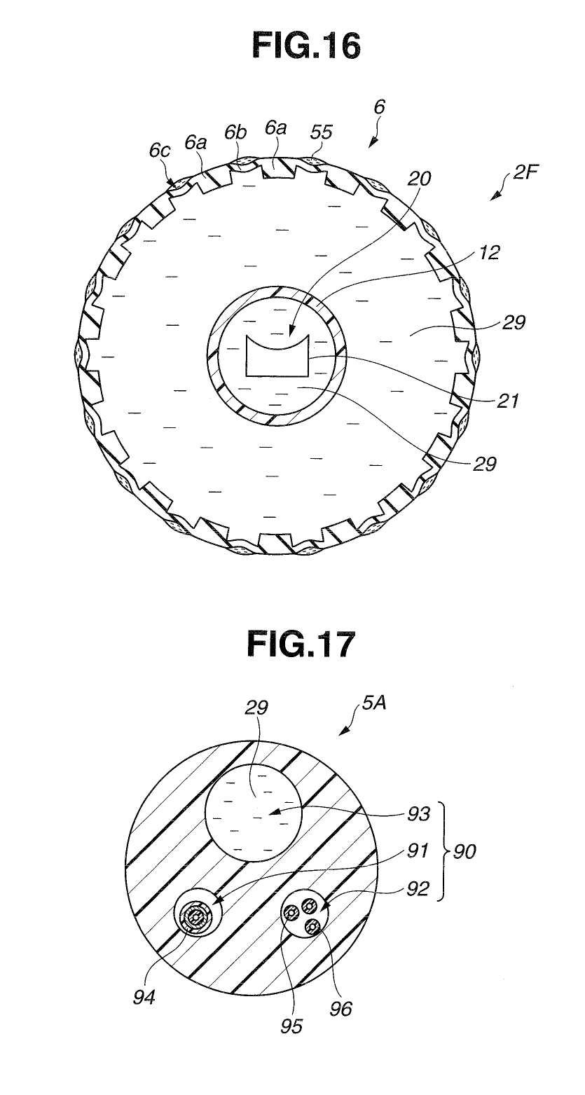

When the operator further pulls the signal cable 5A, however, as shown in FIG. 16, the thinner portions 6b sandwiched between the thicker portions 6a of the balloon 6 are deformed to form recesses, so that a part of the blocking substances 55 enters the deformed portions 6c. In the situation, a further retraction of the ultrasound capsule 2F to the operator's side causes parts of the blocking substances 55 to continuously enter the deformed portions 6c, so that the blocking substances 55 are shunted to the distal end side of the balloon 6.

This allows the ultrasound capsule 2F having the inflated balloon 6, when moving to the operator's side, to be smoothly retracted to the operator's side without being impeded by the blocking substances 55 piled on the proximal end side of the balloon 6. After determining that the movement of the ultrasound capsule 2F to the target site is completed, the operator implements an ultrasound observation again.

After the ultrasound diagnosis, when the ultrasound capsule 2F is taken out through the oral cavity, a predetermined amount of the ultrasound transfer medium 29 is discharged from the balloon 6 to deflate the balloon 6, so that a part of the balloon 6 contacts the narrow wall of tubular body cavity. This allows the ultrasound capsule 2F to be smoothly taken out through the oral cavity because the balloon 6 is drawn to the operator's side with the part of the balloon 6 being in contact with the narrow wall of tubular body cavity.

In this way, in the ultrasound capsule apparatus for wired signal transmission by connecting the ultrasound observation apparatus to an ultrasound capsule by a signal cable, a balloon having thinner portions and thicker portions that are elongated in the longitudinal axial direction and are alternately arranged in the circumferential direction is attached to a predetermined position on the ultrasound capsule. When the signal cable is pulled to bring the ultrasound capsule near, the configuration allows a part of the blocking substances piled on the operator's side of the inflated balloon to deform the thinner portions of the balloon, so that the blocking substances are shunted to the distal end side of the balloon. Therefore, in the ultrasound capsule apparatus of the present embodiment, in the pull of the signal cable to retract the ultrasound capsule having the inflated balloon, any impediment to the movement by the blocking substances piled on the operator's side of the balloon can be prevented, resulting in that the operation to retract the ultrasound capsule can be smoothly performed.

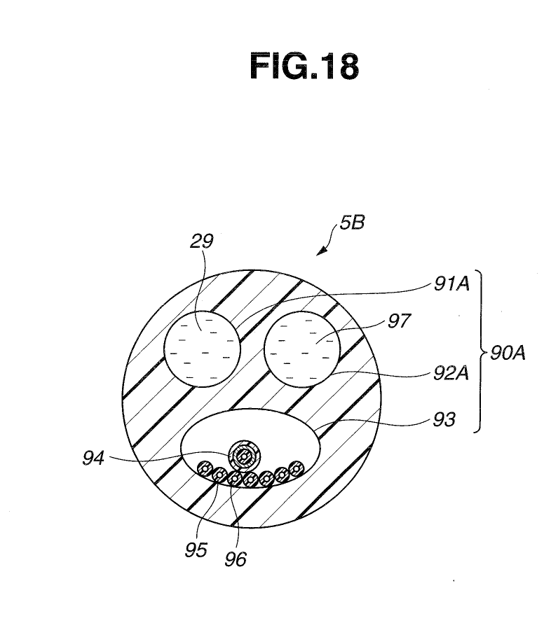

In the above described embodiment, the fluid duct (not shown) is described to be provided in the signal cable 5A, and now the specific configuration will be described below with reference to FIGS. 17 and 18.

With reference to FIGS. 17 and 18, a configuration example of the signal cable having a fluid duct therein will be described below.

As shown in FIG. 17, the signal cable 5A is configured with a multi-lumen tube 90 that includes three lumens 91, 92, and 93, for example. The first lumen 91 is a first cable hole through which a coaxial cable 94 for ultrasound transducer is inserted; the second lumen 92 is a second cable hole through which a cable for encoder 95 and a cable for motor 96 are inserted; and the third lumen 93 is a fluid duct for supplying the ultrasound transfer medium 29.

The signal cable configured with a multi-lumen tube allows the signal cable to have a small diameter.

The signal cable 5A shown in FIG. 17 is configured with the multi-lumen tube 90 including the three lumens for first cable, second cable, and fluid duct 91, 92, and 93 respectively, but the configuration of the multi-lumen tube is not limited to this.

A multi-lumen tube 90A shown in FIG. 18 constitutes a signal cable 5B. The multi-lumen tube 90A includes the three lumens 91A, 92A, and 93A similarly to the multi-lumen tube 90. The first lumen 91A of the multi-lumen tube 90A is a first fluid duct for supplying an ultrasound transfer medium; the second lumen 92A is a second fluid duct for supplying the medical fluid 97 for example; and the third lumen 93A is a cable hole through which cables such as the coaxial cable 94 for ultrasound transducer, the cable for encoder 95, and cable for motor 96 are inserted.

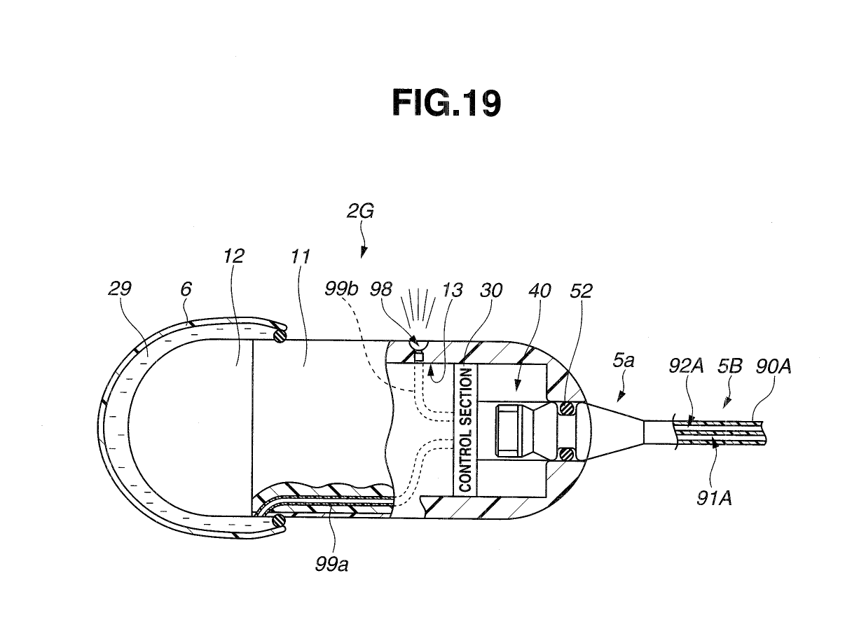

The use of the multi-lumen tube 90A as the signal cable 5B realizes an ultrasound capsule 2G having a configuration shown in FIG. 19.

With reference to FIG. 19, the configuration of an ultrasound capsule of a fifth embodiment will be described below.

As shown in FIG. 19, the ultrasound capsule 2G is provided with the balloon 6. The ultrasound capsule 2G is also provided with a medical fluid ejection port 98. The balloon 6 communicates with one end of a first tube 99a that constitutes a duct for ultrasound transfer medium. The first tube 99a has the other end that communicates with a medium connection port (not shown) provided in the control section 30. The medical fluid ejection port 98 communicates with one end of a second tube 99b that constitutes a medical fluid duct. The second tube 99b has the other end that communicates with a medical fluid connection port (not shown) provided in the control section 30.

The ultrasound observation apparatus 3 of the present embodiment is separately or integrally provided with a pump for supplying the medical fluid 97 and a tank for storing the medical fluid 97, in addition to the pump for supplying the ultrasound transfer medium 29 and the tank for storing the ultrasound transfer medium 29. The balloon 6 is configured to inflate or deflate under the control of a control section (not shown) in the ultrasound observation apparatus 3, and the medical fluid 97 is ejected under the control of a control section in the ultrasound observation apparatus 3.

In the above described embodiment, the signal cable 5 electrically connects the ultrasound capsule 2 to the ultrasound observation apparatus 3. The signal cable 5 is a string member, and is configured to allow the capsule connector 5a at the end on the distal end side of the signal cable 5 to be removably attached to the ultrasound capsule 2.

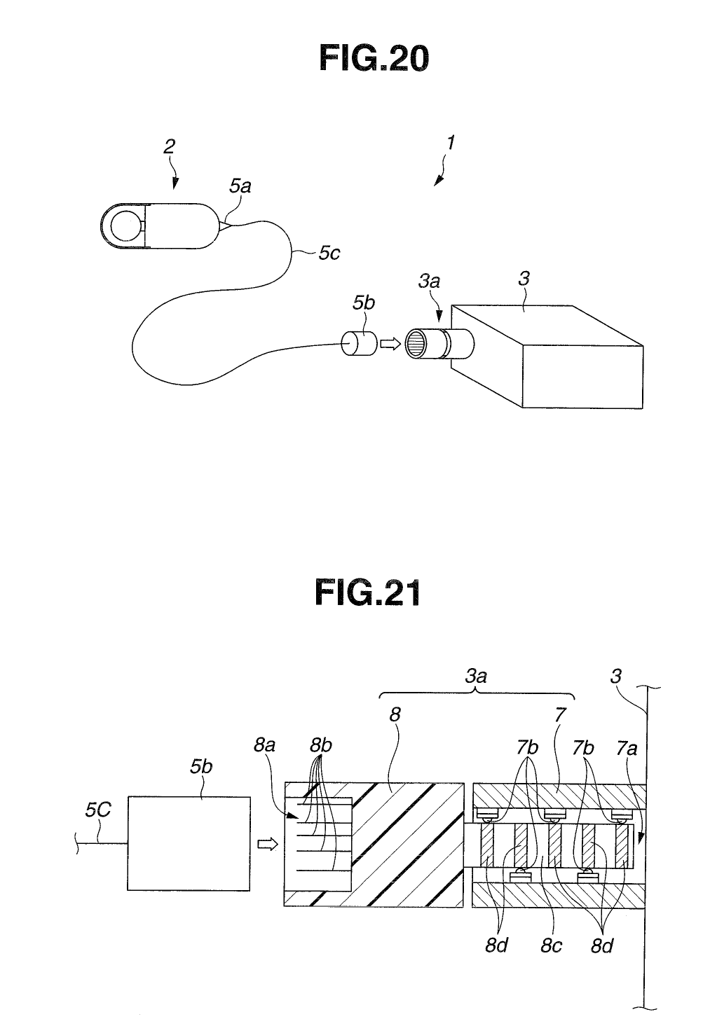

With reference to FIGS. 20 and 21, the configuration of an ultrasound capsule of a sixth embodiment will be described below.

A signal cable 5C of the present embodiment is, as shown in FIG. 20, provided with the capsule connector 5a on the distal end side thereof, and an observation apparatus connector 5b on the proximal end side thereof. The observation apparatus connector 5b is removably coupled to a connector receptacle 3a in the ultrasound observation apparatus 3.

As shown in FIG. 21, the connector receptacle 3a is a rotary connector, and is configured with a connector body 7 and a coupling 8. The coupling 8 has one end in which a recess 8a is provided to accept the observation apparatus connector 5b therein. The recess 8a has a plurality of pins 8b extended therefrom that are inserted into pin holes (not shown) provided in the observation apparatus connector 5b. The coupling 8 has the other end from which a projection 8c extends. The projection 8c has an outer peripheral surface on which a plurality of electrodes 8d are circumferentially provided at a predetermined intervals corresponding to pins 8b. The pins 8b and the electrodes 8d are electrically connected to each other.

The connector body 7 is integrated with a housing that provides the ultrasound observation apparatus 3. The connector body 7 is cylindrical for example, and includes an inner space 7a in which the projection 8c is rotatably and axially supported via a ball bearing (not shown). The connector body 7 has an inner peripheral surface on which a plurality of brushes 7b are provided at predetermined positions. Each of the brushes 7b electrically contacts a corresponding electrode 8d among the electrodes 8d provided to the projection 8c by a predetermined bias.

Thus, when the signal cable 5C is twisted with the observation apparatus connector 5b of the signal cable 5C being connected to the coupling 8 of the connector receptacle 3 a provided in the ultrasound observation apparatus 3, the signal cable 5C and the coupling 8 integrally rotate in the twisted direction. At the time, the brushes 7b electrically contact the electrodes 8d.

Therefore, when the rotating ultrasound capsule 2 is introduced into a body cavity with the ultrasound capsule 2 and the ultrasound observation apparatus 3 being coupled by the signal cable 5C, the signal cable SC is not twisted, but rotates in the direction as the direction of the rotating ultrasound capsule 2.

In this way, the connector receptacle in the ultrasound observation apparatus configured with a rotary connector prevents an application of twist load to the signal cable generated by the rotation of the ultrasound capsule. Therefore, the problem such as cutting of signal line in the signal cable due to the twisting of the ultrasound capsule introduced in a body cavity can be solved.

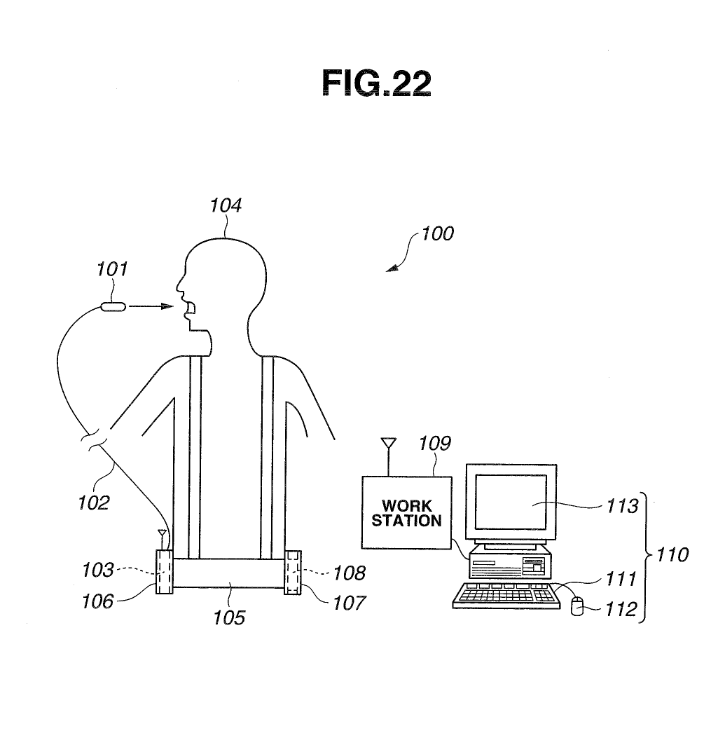

With reference to FIG. 22, the configuration of an ultrasound capsule of a seventh embodiment will be described below.

In an ultrasound observation with the above described ultrasound capsule apparatus, because the signal cable extending from the ultrasound capsule is connected to the ultrasound observation apparatus, there is a problem that the movement of a patient is restricted during the ultrasound observation.

In an ultrasound capsule apparatus 100 shown in FIG. 22, a signal cable 102 extending from an ultrasound capsule 101 is connected to a data storage apparatus 103 having a transmission function. The data storage apparatus 103 is received in a first receiving space 106 provided in a belt for example which a patient 104 puts on. The belt 105 includes a second receiving space 107 in which a battery 108 is received for example. The battery 108 supplies electric power to the data storage apparatus 103.

Also, the transmission function provided to the data storage apparatus 103 allows the ultrasound data recorded in the data storage apparatus 103 to be serially transmitted to a work station 109 that is a receiver. That is, in the ultrasound capsule apparatus 100, the ultrasound data obtained by the ultrasound capsule 2 is wirelessly sent to the work station 109 installed at a position away from the patient, and stored.

The work station 109 is connected to a personal computer (hereinafter, simply referred to as PC) 110 for example. The PC 110 is provided with a keyboard 111 and a mouse 112 as an operation panel for data input, and a display section 113 such as a liquid crystal panel as a display apparatus.

The connection between the PC 110 and the work station 109 enables the transfer of the ultrasound data stored in the work station 109 to a memory medium (not shown) in the PC 100 and a display of an ultrasound tomographic image on a display section 113 after a processing for display.

In this way, the signal cable extending from. the ultrasound capsule is connected at one end of the signal cable to a data storage apparatus that has transmission function and is received in a receiving space in the belt a patient puts on. This allows the patient to freely move within an area where the communication with a work station is established. Thus, some burden to the patient during an ultrasound test can be reduced. If a compact work station can be received in the second receiving space 107, the burden to the patient can be further reduced.

In the above described embodiments, an example in which a wired capsule-type medical apparatus is applied to an ultrasound capsule has been described, but the present invention is not limited to the example, and the above configuration may be applied to other various types of wired medical capsule-type medical apparatuses. Moreover, the present invention is not limited to medical apparatuses, and may be applied to various wired capsule-type examination apparatuses in the industrial field.

The present invention is not limited only to the above described embodiments, but various modifications can be made without departing from the scope of the invention.

What is claimed is:

1. A capsule-type medical apparatus, comprising:
a capsule to be introduced into a tubular body cavity, the capsule including a shunting section for shunting blocking substances that impede the movement of the capsule when the string member is pulled to pull the capsule; and
a string member that extends from one end of the capsule and is able to be pulled when the capsule is pulled.

2. The capsule-type medical apparatus according to claim 1, wherein
the shunting section is a groove that is provided in an outer surface of the capsule to cause blocking substances to pass therethrough, the blocking substances being piled in the moving direction of the pulled capsule when the string member is pulled, and impeding the movement.

3. The capsule-type medical apparatus according to claim 1, wherein
the shunting section is a spiral cover that is rotatably arranged on the outer surface side of the capsule and disposes the blocking substances attached to a wall of the tubular body cavity and is provided with a spiral groove which the blocking substances pass through.

4. The capsule-type medical apparatus according to claim 1, wherein
the shunting section is an oscillation generating apparatus that is provided in the capsule and causes the capsule to oscillate.

5. A capsule-type medical apparatus, comprising:
a capsule to be introduced into a tubular body cavity;
a balloon arranged at a predetermined position outside of the capsule, the balloon including a shunting section for shunting blocking substances that impede the movement of the balloon when the string member is pulled to pull the capsule with the balloon being in close contact with a wall of a tubular body cavity; and
a string member that extends from one end of the capsule and is able to be pulled when the capsule is pulled.

6. The capsule-type medical apparatus according to claim 5, wherein
the balloon includes circumferentially and alternately arranged thicker portions and thinner portions, and
the shunting section is a longitudinal groove that is formed by the thinner portions in the outer surface of the balloon when blocking substances are piled in the moving direction of the balloon that is pulled together with the capsule in pulling the string member.
